(19) United States
(12) Patent Application Publication
(10) Pub. No.: US 2004/0209849 A1
(43) Pub. Date: Oct. 21, 2004

(54) Use of effectors of the central cholinergic nervous system for treatment of delirium

(75) Inventor: Peter Fischer, Wien (AT)

Correspondence Address: YOUNG & THOMPSON, 745 SOUTH 23RD STREET 2ND FLOOR, ARLINGTON, VA, 22202

(73) Assignee: SANOCHEMIA PHARMAZEUTIKA AKTIENGESELLSCHAFT, WIEN (AT)

(21) Appl. No.: 10/757,545

(22) Filed: Jan. 15, 2004

Related U.S. Application Data
(63) Continuation of application No. 09/856,352, filed on Oct. 17, 2001, now abandoned.
Application No. 09/856,352 is a 371 of international application No. PCT/AT99/00284, filed on Nov. 22, 1999.

(30) Foreign Application Priority Data
Nov. 27, 1998 (AT) A1995/98

(51) Int. Cl. A61K 31/66

(52) U.S. Cl. 514/114

(57) ABSTRACT

The invention relates to the use of effectors of the central cholinergic nervous system for the production of a preparation to treat non-anticholinergic delirium.

### CROSS REFERENCE TO RELATED APPLICATIONS

This application is a Continuation of co-pending application Ser. No. 09/856,352, filed on Oct. 17, 2001, application Ser. No. 09/856,352 is the national phase of PCT International Application No. PCT/AT99/00284 filed on Nov. 22, 1999 under 35 U.S.C. § 371. The entire contents of each of the above-identified applications are hereby incorporated by reference.

### BACKGROUND OF THE INVENTION

Acetylcholine ((2-acetoxyethyl)-trimethylammonium-hydroxide) is the neurotransmitter of the cholinergic synapses. Cholinergic synapses can be found in all paths of the parasympathetic nervous system, on the preganglionic sympathetic fibers, the peripheral motor neurons and in almost all regions of the central nervous system (on all cholinergic synapses of the central nervous system).

Acetylcholine is formed in the axon in the vicinity of the presynaptic membrane by the enzyme choline-O-acetyl transferase by acetylation of choline and is stored in the synaptic vesicles. When the presynaptic nerve cell is excited Ca ions flow in and cause release of acetylcholine into the synaptic gap. On the postsynaptic membrane acetylcholine bonds to special receptors (“acetylcholine receptors”), by which signal transmission to the postsynaptic cell is achieved. Postsynaptic cells can be for example another nerve cell, a muscle cell or a glandular cell. Breakdown of acetylcholine into choline and acetate by the enzyme acetylcholine esterase causes the termination of the excitation state and re-establishment of excitation readiness. Choline can then be absorbed again by the presynaptic cell and re-used.

Among the acetylcholine receptors fundamentally two types are distinguished; based on their different reaction to the agonists muscarine and nicotine they are called nicotinic and muscarinic act receptors.

Nicotinic receptors are located on the sympathetic and parasympathetic ganglia, on the neuromuscular end plates and also in the brain. Their occurrence in the brain is associated with learning, the effect of nicotine on the ability to learn and with Alzheimer's disease. The nicotinic acetylcholine receptor is considered the most heavily studied receptor of a neurotransmitter and consists of five polypeptide chains (2 α, one β, γ, and δ chain each) which form a pentagonal pore in the membrane. This pore acts as a cation channel which is opened as soon as the acetylcholine is bound to the two α subunits. Various snake poisons (for example alpha-bungarotoxin, cobra toxin), the arrow poison curare (+)-tubocurarine), lophotoxin and some quaternary ammonium compounds which can bind instead of acetylcholine act as inhibitors of this ion channel. There is a still undetermined number (but at least 3) subtypes of the nicotinic acetylcholine receptor.

Muscarinic receptors consist of a single polypeptide chain and exert their influence via G proteins and various effectors, such as phospholipase C or adenylate cyclase. Five subtypes are pharmacologically defined, in the central nervous system mainly M₁ and M₄ and only in small amounts M₂ and M₃ and M₅ receptors occurring. The excitory effect of the M₁ and M₃ receptors and their location in the hippocampus promise positive cognitive effects when these receptors are activated, while an inhibitory effect is achieved via the M₂ receptor and improvement of cognitive deficits cannot be expected. Conversely, antagonists on the M₂ receptors would also activate the cholinergic central nervous system.

Pharmaceuticals which have an excitation-transferring effect which is comparable to acetylcholine are called cholinergics or parasympathicomimetics and those which oppose these processes, therefore for example inhibit the acetylcholine receptors, are called anticholinergics or parasympathicolytics. Acetylcholine esterase consists of 4 identical subunits and is part of the group of serine esterases. One mole of this enzyme hydrolyzes roughly 25000 moles of acetylcholine per second. This extremely high reaction rate is essential for prompt termination of the nerve pulses transmitted by acetylcholine (in 0.1 ms all the acetylcholine present in the synaptic gap is hydrolyzed). Known (reversible) inhibitors of acetylcholine esterase are demecarium bromide, neostigmine, pyridostigmine bromide and physostigmine and related carbaminic acid esters. Irreversible inhibition is caused also by diisopropyl fluorophosphate, parathion (=E605), tetrastigmine or by organophosphorus compounds (which are used in part as war gases or pest control agents, for example phosgene, alkylphosphates, etc.).

These parasympathicomimetics include besides acetylcholine esterase inhibitors also cholinesters such as (beside acetylcholine) bethanecol, carbachol and methacholine, and alkaloids such as muscarine and pilocarpine. Furthermore the cholinesterase inhibitors physostigmine and galanthamine, but also for example (+)-2-methyl piperidines, act as channel activators on the nicotinic acetylcholine receptors. But there are also nicotinic receptor agonists (nicotine, cytisines, lobelines, anatoxin-a, epibatidines, 2,4 dimethyl-cinnamylidene anabasines, 2,4-dimethyloxybenzylidene anabasine and ABT-418, the isoxazol isosters of nicotine) and muscarinic receptor agonists (R586, arecolines, oxotremorines, AF102B, azaspirodecanes, etc.).

For a few years drugs have been available for the treatment of dementia of the Alzheimers type; they counteract the central nervous shortage of acetylcholine in this condition and thus improve brain performance. In Austria, for this indication the substances galanthamine (Nivalin®), tacrine (Cognex®), donepezil (Aricept®) and rivastigmine (Exelon®) have been approved. Other central inhibitors of acetylcholine esterase are under development worldwide (for example Metrifonate®, Bayer Corp.), Synapton®-Physostigmine slow release, Forest Laboratories; Galantamine-Reminyl, Janssen Pharmaceutica; Eptastigmine, Mediolanum; Velnacrine, Hoechst-Roussel; Suronacrine, Hoechst-Roussel; Huperzine A, Chinese Academy of Science; NX-066, Astra Arcus; KA-672, Schwabe, etc.). Furthermore, at present a series of agonists of the cholinergic system, especially M1 agonists, are being clinically or preclinically tested in their effect for dementia of the Alzheimer's type (for example Memric®, SmithKline Beecham; Talsaclidine®, Boehringer Ingelheim Pharmaceuticls; etc). The inhibitor of acetylcholine esterase which can be administered parenterally, physostigmine, has been approved for decades in almost all countries around the world as an antidote for poisoning with anticholinergic substances (atropine, antidepressants, neuroleptics, . . . ) in ampule form; in Austria as anticholium ampules with the indication “antidote for poisoning, central anticholinergic syndrome”.

In addition to the dementias, therefore chronic organically induced mental disorders, there are the acute organic psychoses which have been called by various names over the history of psychiatry. At present both international diagnosis systems of psychiatric diseases speak of an acute state of confusion and use the term delirium synonymously. The incidence of acute states of confusion is significantly greater than that of the dementias. The occurrence of an acute state of confusion is the result of acute impairment of the function of the central nervous system and can occur at any age even without structural damage to the brain tissue. While an advanced age is a risk factor for the occurrence of acute states of confusion, only roughly 25% of the patients with acute states of confusion in in-patient hospital wards also suffer from a dementia condition (Erkinjuntti et al (1986) Arch Int Med 146: 1923-1926). Prior brain damage as exists in demented patients increases the probability of developing a delirium in an acute function disorder. The causes of an acute state of confusion are numerous.

The most frequent acute states of confusion currently occur in in-patient and surgical wards as the result of internal diseases or after trauma or after surgery. Furthermore, acute states of confusion also occur after non-anticholinergic poisoning (for example by lithium, see the described individual case) and in substance withdrawal (alcohol withdrawal, tranquilizer withdrawal). Acute states of confusion prolong hospital stays, increase the complication rates of the underlying conditions, and increase the mortality of these conditions. They are currently treated on the one hand by the attempt to eliminate the cause (for example, an increase of blood pressure, administering antibiotics for pneumonia, . . . ) and for self-endangerment or endangerment of others in confused and often excited patients by administering high strength neuroleptics which for their part are accompanied by numerous side effects.

Generally, anticholinergic delirium is defined as a delirium which occurs when anticholinergically acting substances are administered. Non-anticholinergic deliria are conversely deliria which occur without anticholinergically acting substances being administered beforehand (therefore generally 48-72 h prior).

In the treatment of deliria at present mainly the treatment of the organic cause is in the foreground. The determination of the cause to which then the (pharmaceutical) treatment of deliria is tailored however due to the plurality of possible causes is extremely complex and time-consuming so that prompt treatment is generally difficult. Generally, first a drug-induced delirium is assumed and therefore initially any non-vitally induced and in any case any anticholinergic medication, and medications which pass through the blood-brain barrier, are discontinued. In addition, in a vigorously hallucinating delirious patient who disrupts the nighttime rest of other patients he must be treated symptomatically with high-strength neuroleptics (for example haloperidol). This neuroleptic medication of the delirious patent must be checked anew each day with regard to indication and dose.

On the other hand, the acute states of confusion however have a higher incidence than even demential and depressive syndromes in the elderly (thus in 30 to 50% of patients older than 70 at any time during in-patient hospitalization, acute states of confusion are observed) so that there is a great demand here for prompt, universally usable treatment.

Overall it is estimated that roughly 10% of in-patient or surgically hospitalized patients during their stay in the hospital at some time experience an episode of delirium (Lipowski, JAMA 258 (1987), 1789-1792).

### SUMMARY OF THE INVENTION

The object of this invention is therefore to make available treatment of non-anticholinergic states of delirium, which can be promptly and universally used without the need for extremely complex study of causes, so that even acute states of confusion can be quickly and reliably improved, cured or prevented.

This object of improving, curing or preventing delirium is achieved by using drugs for treatment of these non-anticholinergic deliria which directly or indirectly increase the activity of the cholinergic central nervous system, for example acetylcholine esterase inhibitors, cholinomimetic postsynaptic receptor agonists, especially on the M₁ and M₃ receptors, nicotinic postsynaptic receptor agonists, autoreceptor antagonists in the cholinergic system, acetylcholine receptor channel activators, M₂ receptor antagonists and combinations of these substances.

### DETAILED DESCRIPTION OF THE INVENTION

The subject matter of this invention is therefore the use of effectors of the cholinergic central nervous system for producing a preparation for treatment or prevention of non-anticholinergic delirium. Surprisingly, acute states of confusion can be promptly and effectively controlled by the treatment as claimed in the invention, although they do not seem to have any primary connection to non-anticholinergic deliria. While therefore for a long time, solely when a drug-induced anticholinergic delirium was suspected, was the cholinesterase inhibitor physostigmine which is approved for this purpose administered parenterally (this leads to usually good clearance of the anticholinergic delirium and improvement of the orientation of the patient), the administration of these substances for non-anticholinergic deliria was for a long time not considered, especially because to date no relationship of the deliria treated as claimed in the invention to the cholinergic system had been assumed.

Effectors of the cholinergic central nervous system are defined as claimed in the invention as medications which lead to intensified activity of the cholinergic synapses by releasing more acetylcholine (for example, aminopyridine), by breaking down less acetylcholine (or breaking down the acetylcholine more slowly) or by exciting central cholinergic synapses (for example, muscarinic and/or nicotinic agonists) or by intensifying the anticholinergic effect on these synapses (acetylcholine-receptor channel activators) or by autoreceptor agonists of the cholinergic system.

Thus, as claimed in the invention, patients after long surgeries with blood loss, intraoperative hypoxia, presurgically poor cognitive status or very old age, therefore patients in whom risk factors are present for the occurrence of a postsurgical delirium, are also treated after their surgery with activators of the cholinergic system in order to not allow the development of the delirium (secondary prevention of delirium).

With this invention however it was possible to prove that pathophysiologically most acute states of confusion are probably based on a functional imbalance of different transmitter systems, probably a relative predominance of monaminergic systems occurring and the cholinergic system being relatively underactive.

Based on this approach therefore any delirium, not only anticholinergic delirium, can be controlled, shortened or cured by a cholinomimetic therapy. This has been impressively confirmed in the observation of individual cases.

This invention relates therefore to treatment of acute states of confusion of any type and cause by means of pharmacologic activation of the cholinergic system, for example by modern acetylcholine esterase inhibitors such as rivastigmine, galanthamine, tacrine, or donepazil or follow-up preparations with direct or indirect cholinomimetic site of action. While this therapy in anticholinergic delirium is obvious on the basis of existing treatment practice which was always directed at control of the causes, in all other deliria it is a completely new therapeutic indication of these drugs which have been developed or are under development for treatment of dementia of the Alzheimer type.

The definition of an acute state of confusion or delirium was often unclear in its history. Thus the concept of “organic psychosis” goes back to K. Bonhoeffer who distinguished between acute “exogenous reaction types”, therefore etiologically nonspecific reactions of the brain to acute damage, and chronic exogenous reactions types. The latter are for example the posttraumatic syndrome, therefore disturbance of memory and thought, or dementia which at the time was diagnosed only for severe irreversible posttraumatic syndrome. If in a further sense therefore all mental disorders as a result of brain damage can be called “organic psychoses”, the concept of “organic psychosis in a narrower sense” is often applied only to acute psychoses with brain damage and thus corresponds to the internationally unconventional historical concept of “acute exogenous reaction type”. Other terms which are used synonymously are “symptomatic psychoses”, “somatically substantiated psychoses” or the concept of “function psychoses” which expresses the reversibility of these state pictures. The hopeful term “transitional syndrome” is also similar to the last concept.

Today these concepts are no longer used for “organic psychoses in a narrower sense”, but only more the modern internationally conventional diagnostic entity “acute state of confusion (delirium)” is used, the scope of which is generally recognized and defined at present.

Thus, the above described “organic psychoses in a narrower sense” in the two currently used modern psychiatric diagnosis systems, therefore DSM-IV (American Psychiatric Association: Diagnostic and Statistical Manual of Mental Disorders, Fourth Edition, Washington D.C., APA, 1994) and the ICD-10 (ICD-10, WHO, International Classification of Mental Disorders, Bern, H. Huber, 1991) are named in this way and have been defined very similarly. Currently the diagnosis system of the American Psychiatric Association APA, the Diagnostic and Statistic Manual DSM in its 4th revision (DSM-IV, 1994) speaks of delirium and divides it into

(1) Delirium based on a medical disease factor, therefore for example, postsurgical delirium, delirium after hypoglycemic coma, delirium after resuscitation, . . . )

(2.1) Substance-induced delirium, subform substance intoxication delirium, therefore for example delirium after intoxication with alcohol, amphetamines, opiates, atropine, etc, and

(2.1) Substance-induced delirium, subform substance withdrawal delirium, therefore delirium in alcohol withdrawal, drug withdrawal, narcotic withdrawal, etc. and

(3) Delirium based on multiple etiologies. When the cause is uncertain,

(4) a “nonspecific delirium” is diagnosed.

In the diagnosis system of the World Health Organization WHO, ICD 10 (1991), organic mental disorders, besides the dementias, are defined as “delirium, not caused by alcohol or psychotropic substances.

Since a delirium more probably occurs in any basic cerebro-organic disease, there are always patients again and again who suffer from a dementia which is suddenly complicated by a delirium. Here the DSM-IV first diagnoses dementia and then with an additional number an additionally present delirium, while in the ICD-10 first of all the delirium is diagnosed as “delirium with dementia”. It is important that most of the acute states of confusion are not linked to a dementia condition.

Clinically, deliria (acute states of confusion)) designate an ordinarily rather suddenly occurring and transitory disorder of intellectual functions with impairment of attention (qualitative and quantitative disturbance of consciousness) still present often accompanied by disruption of the circadian rhythm and psychomotor peculiarities (hypoactive or hyperactive delirium).

Perception, thinking and memory functions are disrupted in part or seriously in acute states of confusion. The acuity of perception decreases, the perceptions which are responsible for the impression of confusion are misinterpreted. In addition to illusionary misjudgments, especially optical hallucinations also occur which encompasses not only simple matters, but also moving individuals, animals, and entire scenarios. When these phenomena occur suddenly, the patients are usually extremely anxious, try to flee or fight against the phenomena.

Thinking is disorganized, thinking and speech incoherent, the ability to cope is lost, abstract concepts can no longer be grasped, the ability of “inner imagining” (e: imagery) is lost. The patients therefore answer randomly and absurdly, without noticing this absurdity themselves. The disorder of perception and thinking together with still disturbed memory function promotes the occurrence of delusional interpretations and delusional certainties. As a result of this intellectual disorder, orientation to time and place, occasionally also to the situation, rarely to the individual, is lost.

Attentiveness is always disrupted; this can also be duplicated in a pathological EEG. The patients can be easily distracted, cannot voluntarily modulate their attention or address new matters. The attention disorder appears for example in disordered repeating of letters and numbers. In the case of slight acute states of confusion the attention fluctuates greatly, so that individual actions are suddenly very good, seconds later they cannot be performed at all. These fluctuations of attention can be easily observed in a concentration test lasting a few minutes (for example, deletion tests). The impairment of attention also exhibits major overlapping with the concept of disordered consciousness. The qualitative consciousness disorder of confusion however can also be accompanied by a only very minor quantitative disorder of consciousness (fatigue).

The alertness of the patient during the day is often reduced, it is especially typical for the patient to doze off again and again during the day, while at night they awaken easily or are completely awake and then especially agitated and nervously excite commotion. In severe cases the circadian rhythm is completely reversed. In these patients increased activity and agitation are especially typical in late afternoon and evening.

Delirious patients can exhibit reduced or enhanced psychomotility which is expressed in gestures and speech. While patients with increased psychomotility are more quickly identified by nursing personnel and relatives, there is the danger that inactive patients will not be diagnosed and treated as a state of confusion.

The mood of a delirious patent can be depressive, but is often dysphoric (grumpy-excitable); this has to do with the increased reactivity to external stimuli. The sympathetic nervous system is overexcitable, vegetative symptoms such as sudoresis, tachycardia, blood pressure and pulse fluctuations, flushing, dilated pupils, anxiety and rage reactions occur.

As claimed in the invention all deliria are therefore treated which are non-anticholinergic, i.e. in which the delirium is not caused by for example anticholinergic intoxication (for example, by belladonna poisoning or poisoning with other tropane alkaloids) or by degeneration of the cholinergic system in degenerative dementia (compare Int. Psychoger. 3(2) (1991), 373-395; Psych. Clin. Neuroscience 248 (Supp. 1) (1998) p. 59).

Preferably a postoperative delirium, a delirium during an internal disease or a posttraumatic delirium is treated as claimed in the invention. Especially in these clinically extremely important deliria there has not been any satisfactory pharmaceutical treatment available for a long time.

Another preferred embodiment of this invention relates to the treatment of deliria which are caused by non-anticholinergic intoxication.

Another application of the invention is the treatment of deliria caused by substance withdrawal, in which likewise an especially great demand for effective treatment methods prevails.

Furthermore, a delirium which is caused by hypoglycemic processes, especially hypoglycemic coma, can be effectively treated as claimed in the invention.

Often deliria also occur in conjunction with resuscitation, which of course can likewise be treated as claimed in the invention.

It is common to all these deliria that they seem not to have any apparent relationship to the cholinergic system, by which their effective treatment with the measures as claimed in the invention is especially surprising.

A differential diagnosis of acute states of confusion (of delirium) is possible from the psychopathology in cross section and from the information about the course from the outside history. There is no specific diagnostic test even if delirium can be precluded with an unremarkable EEG. In addition to dementia, when a delirium is diagnosed schizophrenia, mania, and a psychogenic twilight state must be precluded. In addition, again and again aphasias after stroke especially in the elderly are confused with deliria. Schizophrenic exacerbations, also in the elderly, are often accompanied by auditory and also visual hallucinations. Moreover, in these states which are assumed to be delirious in endogenous psychoses the EEG is generally unremarkable.

The major difference between pure delirium and a condition of dementia is indicated by the history, the delirium beginning acutely and often observed for the first time at night by family members and nursing personnel, while dementia begins insidiously and is discovered in the daily routine. While the state of the demented patient is relatively stable over days and weeks, in delirium major fluctuations occur also within a day and frequent short episodes of good orientation occur. In incipient dementias generally attentiveness is not reduced or these patients act very troubled and wakeful, conversely delirious patients are very distractible and thus act unfocussed.

There is no representative epidemiological study of the frequency of acute states of disorder, but studies on patients admitted to in-patient wards indicate that acute states of disorder have a greater incidence than demential and depressive syndromes themselves among the elderly. Acute states of disorder are observed in 30 to 50% of patients older than 70 at any time during in-patient hospitalization.

Therefore treatment is carried out as claimed in the invention by administering to the patient a suitable dose of an agent which can increase directly or indirectly the activity of the (central) nervous system. Since a large number of these medications are already available on the market for other indications, the dose for the treatment as claimed in the invention can be determined based on the dosages already used. As a result of the good effectiveness of treatment as claimed in the invention the administration of neuroleptics is no longer necessary as claimed in the invention.

Accompanying treatment measures are however useful, as in previous treatment practice: Thus the electrolyte and fluid balance should be monitored and nutrition and supply of the necessary vitamins and trace elements should be ensured.

Nursing personnel trained in reality orientation demonstrably accelerate the reorientation of delirious elderly patients. Patients should be kept in quiet, well lighted spaces, should have familiar objects around them and also have a clock and calendar as well as newspapers. The patients should be addressed repeatedly about their sense of orientation and should be verbally reoriented.

By definition delirium is a transitory condition which generally lasts only a few days to a few weeks. But, due to the concomitant massive vegetative symptomatics in multimorbid elderly patients, delirium is a dangerous disease complication. Not only is the circulatory system burdened, with the result of an impaired cardiac situation, but in the state of confusion also self-mutilation and injuries are possible. In addition, delirious patients remove intravenous catheters, open dressings and fall out of bed at night especially easily. If patients are too heavily sedated, deep leg venous thromboses and pulmonary embolisms are a threat. Overall the mortality of delirium is scientifically difficult to estimate because the delirious states often occur accompanied by serious extracerebral conditions and the mortality of these conditions then cannot be separated from the mortality of the delirium. Results from the Lavkoff working group (Arch. Inter. Med 152 (1992), 334-40) suggest that numerous patients after suffering an acute state of confusion can suffer them again; complete restoration of health therefore does not occur in all patients, as hoped. This persistence of symptoms is currently the topic of international research and is unclear in its pathophysiology.

Surprisingly, at present there is no internationally recognized theory of conditions of cerebral origin for a delirious syndrome. In particular the question is unanswered whether a delirium is the result of a diffuse brain function disorder or a localized disorder, for example in our system of attentiveness. Influenced by the ease of triggering delirium by anticholinergically acting substances, there is also a biochemical theory of imbalance of the centrally cholinergic and monaminergic (noradrenergic and/or serotonergic and/or dopaminergic) mechanisms as the basis of the delirium. Probably the numerous syndromes of delirium are based on different pathogenetic mechanisms in systems which can be easily localized in part, in part as the result of diffuse cerebral disorders. While delirious syndromes after hypoglycemias, after cardiac irregularities or drops of blood pressure certainly are an expression of functional disorders of nerve cells and the neural system in wide parts of the cerebrum, other syndromes of delirium, for example Korsakoff psychosis, are an expression of localized lesions in the brain stem and the interbrain.

An entirely different approach to the pathogenesis of states of confusion is provided by the concept of homeostasis. It is a gerontological principle that one major effect of the aging of a living organism is its adaptability to changes in the environment becoming continuously less, as occurs in the case of the brain by medications, hypoglycemias, and focal lesions.

The structures and functions of parts of the organism in various phases of life have different flexibility within which the normal range of functioning can be maintained under stress. This adaptability is the result of a host of individual reactions, for example, at the synapse level, which are usually analyzed in isolation by science. Nerve cells, the neural system, the brain and our bodies at any time are in a dynamic and mutually interdependent state which is called homeostasis and is a steady state equilibrium which can adjust to disruptions only within a certain range. One such steady state guarantees continuity, but also yields the possibility of adapting. Processes which are used to maintain homeostasis are systems of adaptivity or compensation.

With increasing age it becomes more difficult to control homeostasis, the bandwidth of the normal range becomes narrower, the stability and capacity to compensate become less. This is expressed in the growing susceptibility to impairment which means delirium in the functions of consciousness, alertness, attentiveness or orientation. A rather thoroughly studied example of a central function which is less adaptable over age is thermoregulation; on its example the age-dependent limitation of adaptability of brain function can be shown. The consequence of this impaired adaptivity is also sensitivity to psychotropic drugs which is clearly increased with increasing age.

The fluctuation of the equilibrium of various transmitter systems of our brain, a fluctuation which can no longer be compensated within a time framework, whether by psychotropic drugs or other disruptive influences, leads to collapse of the highest plane of cerebral homeostasis, the processing of conscious thoughts. Consequently deliria do not occur in slow acting impairments of the function or structure of the brain, but are observed in very sudden changes of the level of brain function.

As follows from the aforementioned, a delirium can be due to innumerable factors. Differentiation of primarily cerebral and secondarily cerebral causes and prompt recognition of easily treatable causes such as drug side effects, disorders of water and electrolyte balance, vitamin deficiencies and cardiovascular disorders is clinically important. The most important risk factor for occurrence of delirium is age. The following table outlines the most important causes of acute confusion.

TABLE
Most important causes of acute states of confusion

1. Primary cerebral
skull-brain trauma
primarily degenerative diseases
compressive processes
inflammatory processes
vascular-ischemic processes
intoxications (not only anticholinergic)
O₂ deficiency (anemia, respirator insufficiency.
2. Secondary cerebral
postoperative
after anesthesia (also without surgery)
intoxications
drug side effects
cardiovascular impairments
metabolic-toxic (kidneys, liver, . . . )
water and electrolyte balance
endocrine disorders (thyroid, parathyroid)
deficiency of vitamins or trace elements
infections
thermoregulation disorders
impairments of the sense organs
stress

Postoperative delirium is one of the main applications of the therapy as claimed in the invention and will therefore be briefly described. Due to the increase in complex surgeries in the area of heart and thoracic surgery, neurosurgery, transplant surgery, but also emergency surgery, reconstructive surgery, etc., in spite of improvements in surgical techniques and anesthesia, postoperative states of confusion occur more and more. On the one hand, the better techniques reduce the incidence of these feared postsurgical complications, on the other hand due to the improved techniques more and more seriously ill, multimorbid and also older and older individuals undergo these surgeries. In spite of the great clinical importance, these states of confusion are less studied since this psychiatric condition is treated only quite rarely in psychiatric hospitals. The incidence of postoperative delirium, depending on the study and the target patient population or operation, fluctuates between 0% and 73.5% (Dyer et al., Postoperative Delirium. Archives of Internal Medicine, 155 (13) (1995) 461-465). The occurrence of postoperative delirium is a risk factor for increased complication rates and increased mortality, it increases the probability of home nursing care, prolongs hospital stays, more and thus more expensive readmissions and the postoperative state of confusion can have long-lasting mental consequences.

One example of the frequency of postoperative states of confusion can be found in orthopedics. In the 3 prospective studies carried out worldwide to date, between 18 and 41% of patients who underwent a hip replacement as scheduled suffered this complication. In 4 prospective studies on patients with hip replacement after fracture of the femoral neck, therefore in unscheduled surgery after trauma. the frequencies of this psychiatric complication were 26% to 52% of patients!

The invention is detailed using the following example to which it is of course not limited.

### EXAMPLE

Treatment of a non-anticholinergic delirium with an orally administered modern inhibitor of acetylcholine esterase, rivastigmine

A 70 year old patient had suffered from manic-depressive disease since age 35. Until recently there were no mental disintegration phenomena. After a urinary tract infection with lithium therapy of the manic-depressive condition, lithium poisoning occurred with a severe hyperactive delirium which forced the patient to be admitted to a hospital on an in-patient ward. These lithium poisoning deliria generally subside completely only after 6-8 weeks. The patient was transferred to a private clinic after 4 days and was highly psychotic, urinated in the corner of the room, talked to the lamp on the night table, hallucinated strange individuals and dwarves in the room, repeatedly fled the ward, believed that the nursing personnel wanted to kill her, was extremely agitated in motor response and restless, fumbled around with the blanket and phone cord, fell asleep repeatedly during the day, but remained up all night. The patient cried and laughed in alternation, suddenly became afraid again and again and then laughed. All these symptoms were very unstable, highly varied, but the patient was not actually responsive on the admission date. On the Trzepacz scale of delirium (Psychiatry Research, 23, (1988), 89-87) a score of 29 (of a possible 32) corresponding to a severe delirium was reached. After 48 hours of therapy with Exelon® (rivastigmine) at a dose of 2×1.5 mg the patient was oriented, no longer hallucinated, did recall her delusionary ideas, but distanced herself from them, but was still slightly disturbed, slightly unfocussed, was still sleepy during the day, but at night there were only more discrete episodes of sleeplessness. The symptoms were stable at this point without special fluctuations, the delirium score dropped to 8 within 48 hours. After 4 weeks rivastigmine (Exelon) was discontinued and even now fluctuations of attentiveness, sleep disorders, agitation and daily fluctuations still occur for 2-3 weeks, as could have been expected after this time in the spontaneous course of lithium poisoning.

1-6. (canceled)

7. A method of treating a patient diagnosed with a non-anticholinergic state of delirium, comprising:
administering to said patient in need thereof an effective amount of an acetylcholine esterase inhibitor to treat said delirium, and wherein said delirium occurs without anticholinergically acting substances being administered beforehand.

8. A method of treating a patient diagnosed with a non-anticholinergic state of delirium, comprising:
administering to said patient an effective amount of an acetylcholine esterase inhibitor to treat said delirium.

9. The method according to claim 8, wherein said delirium is caused by postoperative complications or from the effects of an internal disease.

10. The method according to claim 8, wherein said delirium is caused by a hypoglycemic process.

11. The method according to claim 10, wherein the hypoglycemic process is a hypoglycemic coma.

12. The method according to claim 8, wherein the delirium is caused by complications arising from the resuscitation of a patient.

13. The method according to claim 8, wherein said acetylcholine esterase inhibitor is selected from the group consisting of rivastigmine, galantamine, tacrine and donepezil.

14. The method according to claim 8, wherein said acetylcholine esterase inhibitor is rivastigmine.

15. The method according to claim 8, wherein said delirium is caused by a non-anticholinergic substance withdrawal.

16. The method according to claim 8, wherein said delirium is caused by non-anticholinergic intoxication.
